SEQUENCE LISTING (16 sequences)

SEQ ID NO: 1 - DNA, 2616 bp, synthetic construct
  source 1..2616: mol_type = other DNA
atgtctccaccgctcctgggtgcggaggaaggaggaacaagcgatgtcaccaaagttgctggttctccctcctcaaaggactcccctgatggccttggcttgggcctgaaagagaggaactaccttggactttccgactgttcatcggttgacagttcaacggtgtcgggccagtcagatcctgaggcccagaacaacaagctgaacctgaaggccacagagctcaggcttgggcttccaggatctcagtccccggaaagagatcttgatgagaagcctctgttccctctccttccctcaaaggatgggatcaagactgtcgtctcaggcaataagaggggtttctctgatgcaattgatgggttctcggataagaagtggatgtttcactctgccgggactgatgagggcacacagccattgggacagggtaagtttcctgctaatgctggccctaagcagccagctaccaaagaggtgccggcaaagcccctgcctgaacgagcaaatgggattaacccgagcagagctgcgaacaatgcaccagctgccaaggcccaggtcattggttggcctccagtgagatccttcagaaagaacactttggcgactacttctaaggataatgatgaagtagatggaaaacctggtcctggtgctctcttcatcaaggtgagcatggatggtgctccatatttgcggaaggtggacctaaggaactactccaagtactatgaactctccgttgctctcgagaagatgttcagcggttttaccatagggcaatgtggaactaatggaactggtggaagggaatcgatgagcgagagcaagctgaaggatcttctgcatgggtcagactatgttctcacatacgaggacaaagatggtgattggatgctagttggagatgtcccatgggaatgcccccttccccccctcttggccgcctctgcgctctcaggaatcgagatctcggatcgtcgcgcctctgcttcatcgattcagctcgccgcgatctctcagttctctttggtaagatttcaagagattggctcaagaatgcgaggatacgaagatgatgagtacgaggattatgataattatgaggaggaagggtacgagcaagaggaaggcggtgatgaagaagatgaatatgaagaggaagaagaagagccaaagcccaccaaggaggaattggaatatttggagttgaggcagaaactgaaggaatctatcaggaagcggatgaagaaggcagcttctggttcagaagagataaagaaagacaattatggctctttctttggcccttctaaacctgtcattgcacagcgagtgattcaagagagcaagtcattgttggagaatcaacatttggctgcccgattgttgaagtcccatcagaatggtaataagagttcttcttcaagacctgatggttccaagcctggagtgcgtccccaagctcccagagtaagccagctgaaacagagagttcagaaaataaaagatacaagggactactcttttctattatctgatgatgctgagctccctgcctcgcccaaacaacctccaccacgaagtatccctgctcccagtttggatgctcgctcagctaaattatcgtctcagagcagacttaacccagggagcaatagcagacatgtaaatggtggtcgtgaagaaagaagaccagcttcatctaatcgtcacagtaaccctagaccaggctcctctaatggtcacagtaataatagacctggatctgctaatggttatagcaacaccagaccaggctctgctaatggtcacagtaacctcagaccaggctccgctaatgttcacagtaacctcagaccaggctccgctaatggtcacagtaacctcagaccaggctccgccaatggtcacagtaacctcagaccaggctccgccaatggtcacagtaatcttggagccggaccaaataagttgtcttctgctagtaagcctcagatggcagccaataggaagcagctcggtagcaacagtgggaatgggcctggccggcctgcagtttcgaatgggttgcaaccaaagaagccaattccacccccacagagatcttctcaaggtttgaaaagctctgttcctgctggggtgaaaccgccagccacaaagatgcagtcctctgcttcgaagtaccaggtggaaccaagaagaaatgtgcagcaaccaaacaagggcaaaattttatctaaacattcggttgcttcgactagacctcaggttcaagctcagatgagcaaacctcagaagcaaatcccctctcataacagactgcaagatcagaggcccaagaaaaggcccagcagaccattccctgaagacgacgatgacaatgatgcggccatcagtatgatcagaaaaatgtttggatacaatccgcaaaagtttgctggtcgtgatgaagacgacagtgatatggaagcaaactttgacgacatcatgagggaagaaaagaggagtgcgagaatcgcccaaaaggaggacgaggaacagctgcgcttgatagaggaggaggaaaggcgcgaacgcgaacggataagaaaaaagcggaagatgggataa

SEQ ID NO: 2 - DNA, 2616 bp, synthetic construct
  source 1..2616: mol_type = other DNA
atgtctccaccgctcctgggtgcggaggaaggaggaacaagcgatgtcaccaaagttgctggttctccctcctcaaaggactcccctgatggccttggcttgggcctgaaagagaggaactaccttggactttccgactgttcatcggttgacagttcaacggtgtcgggccagtcagatcctgaggcccagaacaacaagctgaacctgaaggccacagagctcaggcttgggcttccaggatctcagtccccggaaagagatcttgatgagaagcctctgttccctctccttccctcaaaggatgggatcaagactgtcgtctcaggcaataagaggggtttctctgatgcaattgatgggttctcggataagaagtggatgtttcactctgccgggactgatgagggcacacagccattgggacagggtaagtttcctgctaatgctggccctaagcagccagctaccaaagaggtgccggcaaagcccctgcctgaacgagcaaatgggattaacccgagcagagctgcgaacaatgcaccagctgccaaggcccaggtcattggttggtctccagtgagatccttcagaaagaacactttggcgactacttctaaggataatgatgaagtagatggaaaacctggtcctggtgctctcttcatcaaggtgagcatggatggtgctccatatttgcggaaggtggacctaaggaactactccaagtactatgaactctccgttgctctcgagaagatgttcagcggttttaccatagggcaatgtggaactaatggaactggtggaagggaatcgatgagcgagagcaagctgaaggatcttctgcatgggtcagactatgttctcacatacgaggacaaagatggtgattggatgctagttggagatgtcccatgggaatgcccccttccccccctcttggccgcctctgcgctctcaggaatcgagatctcggatcgtcgcgcctctgcttcatcgattcagctcgccgcgatctctcagttctctttggtaagatttcaagagattggctcaagaatgcgaggatacgaagatgatgagtacgaggattatgataattatgaggaggaagggtacgagcaagaggaaggcggtgatgaagaagatgaatatgaagaggaagaagaagagccaaagcccaccaaggaggaattggaatatttggagttgaggcagaaactgaaggaatctatcaggaagcggatgaagaaggcagcttctggttcagaagagataaagaaagacaattatggctctttctttggcccttctaaacctgtcattgcacagcgagtgattcaagagagcaagtcattgttggagaatcaacatttggctgcccgattgttgaagtcccatcagaatggtaataagagttcttcttcaagacctgatggttccaagcctggagtgcgtccccaagctcccagagtaagccagctgaaacagagagttcagaaaataaaagatacaagggactactcttttctattatctgatgatgctgagctccctgcctcgcccaaacaacctccaccacgaagtatccctgctcccagtttggatgctcgctcagctaaattatcgtctcagagcagacttaacccagggagcaatagcagacatgtaaatggtggtcgtgaagaaagaagaccagcttcatctaatcgtcacagtaaccctagaccaggctcctctaatggtcacagtaataatagacctggatctgctaatggttatagcaacaccagaccaggctctgctaatggtcacagtaacctcagaccaggctccgctaatgttcacagtaacctcagaccaggctccgctaatggtcacagtaacctcagaccaggctccgccaatggtcacagtaacctcagaccaggctccgccaatggtcacagtaatcttggagccggaccaaataagttgtcttctgctagtaagcctcagatggcagccaataggaagcagctcggtagcaacagtgggaatgggcctggccggcctgcagtttcgaatgggttgcaaccaaagaagccaattccacccccacagagatcttctcaaggtttgaaaagctctgttcctgctggggtgaaaccgccagccacaaagatgcagtcctctgcttcgaagtaccaggtggaaccaagaagaaatgtgcagcaaccaaacaagggcaaaattttatctaaacattcggttgcttcgactagacctcaggttcaagctcagatgagcaaacctcagaagcaaatcccctctcataacagactgcaagatcagaggcccaagaaaaggcccagcagaccattccctgaagacgacgatgacaatgatgcggccatcagtatgatcagaaaaatgtttggatacaatccgcaaaagtttgctggtcgtgatgaagacgacagtgatatggaagcaaactttgacgacatcatgagggaagaaaagaggagtgcgagaatcgcccaaaaggaggacgaggaacagctgcgcttgatagaggaggaggaaaggcgcgaacgcgaacggataagaaaaaagcggaagatgggataa

SEQ ID NO: 3 - protein, 871 aa, synthetic construct
MSPPLLGAEEGGTSDVTKVAGSPSSKDSPDGLGLGLKERNYLGLSDCSSVDSSTVSGQSDPEAQNNKLNLKATELRLGLPGSQSPERDLDEKPLFPLLPSKDGIKTVVSGNKRGFSDAIDGFSDKKWMFHSAGTDEGTQPLGQGKFPANAGPKQPATKEVPAKPLPERANGINPSRAANNAPAAKAQVIGWPPVRSFRKNTLATTSKDNDEVDGKPGPGALFIKVSMDGAPYLRKVDLRNYSKYYELSVALEKMFSGFTIGQCGTNGTGGRESMSESKLKDLLHGSDYVLTYEDKDGDWMLVGDVPWECPLPPLLAASALSGIEISDRRASASSIQLAAISQFSLVRFQEIGSRMRGYEDDEYEDYDNYEEEGYEQEEGGDEEDEYEEEEEEPKPTKEELEYLELRQKLKESIRKRMKKAASGSEEIKKDNYGSFFGPSKPVIAQRVIQESKSLLENQHLAARLLKSHQNGNKSSSSRPDGSKPGVRPQAPRVSQLKQRVQKIKDTRDYSFLLSDDAELPASPKQPPPRSIPAPSLDARSAKLSSQSRLNPGSNSRHVNGGREERRPASSNRHSNPRPGSSNGHSNNRPGSANGYSNTRPGSANGHSNLRPGSANVHSNLRPGSANGHSNLRPGSANGHSNLRPGSANGHSNLGAGPNKLSSASKPQMAANRKQLGSNSGNGPGRPAVSNGLQPKKPIPPPQRSSQGLKSSVPAGVKPPATKMQSSASKYQVEPRRNVQQPNKGKILSKHSVASTRPQVQAQMSKPQKQIPSHNRLQDQRPKKRPSRPFPEDDDDNDAAISMIRKMFGYNPQKFAGRDEDDSDMEANFDDIMREEKRSARIAQKEDEEQLRLIEEEERRERERIRKKRKMG

SEQ ID NO: 4 - protein, 871 aa, synthetic construct
MSPPLLGAEEGGTSDVTKVAGSPSSKDSPDGLGLGLKERNYLGLSDCSSVDSSTVSGQSDPEAQNNKLNLKATELRLGLPGSQSPERDLDEKPLFPLLPSKDGIKTVVSGNKRGFSDAIDGFSDKKWMFHSAGTDEGTQPLGQGKFPANAGPKQPATKEVPAKPLPERANGINPSRAANNAPAAKAQVIGWSPVRSFRKNTLATTSKDNDEVDGKPGPGALFIKVSMDGAPYLRKVDLRNYSKYYELSVALEKMFSGFTIGQCGTNGTGGRESMSESKLKDLLHGSDYVLTYEDKDGDWMLVGDVPWECPLPPLLAASALSGIEISDRRASASSIQLAAISQFSLVRFQEIGSRMRGYEDDEYEDYDNYEEEGYEQEEGGDEEDEYEEEEEEPKPTKEELEYLELRQKLKESIRKRMKKAASGSEEIKKDNYGSFFGPSKPVIAQRVIQESKSLLENQHLAARLLKSHQNGNKSSSSRPDGSKPGVRPQAPRVSQLKQRVQKIKDTRDYSFLLSDDAELPASPKQPPPRSIPAPSLDARSAKLSSQSRLNPGSNSRHVNGGREERRPASSNRHSNPRPGSSNGHSNNRPGSANGYSNTRPGSANGHSNLRPGSANVHSNLRPGSANGHSNLRPGSANGHSNLRPGSANGHSNLGAGPNKLSSASKPQMAANRKQLGSNSGNGPGRPAVSNGLQPKKPIPPPQRSSQGLKSSVPAGVKPPATKMQSSASKYQVEPRRNVQQPNKGKILSKHSVASTRPQVQAQMSKPQKQIPSHNRLQDQRPKKRPSRPFPEDDDDNDAAISMIRKMFGYNPQKFAGRDEDDSDMEANFDDIMREEKRSARIAQKEDEEQLRLIEEEERRERERIRKKRKMG

SEQ ID NO: 5 - DNA, 21 bp, synthetic construct
  source 1..21: mol_type = other DNA
atgtctccaccgctcctgggt

SEQ ID NO: 6 - DNA, 23 bp, synthetic construct
  source 1..23: mol_type = other DNA
tcagtttcttgccttggatttct

SEQ ID NO: 7 - DNA, 35 bp, synthetic construct
  source 1..35: mol_type = other DNA
caggtcattggttggtctccagtgagatccttcag

SEQ ID NO: 8 - DNA, 35 bp, synthetic construct
  source 1..35: mol_type = other DNA
ctgaaggatctcactggagaccaaccaatgacctg

SEQ ID NO: 9 - DNA, 43 bp, synthetic construct
  source 1..43: mol_type = other DNA
cacgggggacgagctcggtaccatgtctccaccgctcctgggt

SEQ ID NO: 10 - DNA, 45 bp, synthetic construct
  source 1..45: mol_type = other DNA
ccatgtcgactctagaggatcctcagtttcttgccttggatttct

SEQ ID NO: 11 - DNA, 20 bp, synthetic construct
  source 1..20: mol_type = other DNA
tattgttggtcgtcccaggc

SEQ ID NO: 12 - DNA, 20 bp, synthetic construct
  source 1..20: mol_type = other DNA
cctctcttcgattgggcctc

SEQ ID NO: 13 - DNA, 23 bp, synthetic construct
  source 1..23: mol_type = other DNA
ggtaacattgtgctcagtggtgg

SEQ ID NO: 14 - DNA, 23 bp, synthetic construct
  source 1..23: mol_type = other DNA
aacgaccttaatcttcatgctgc

SEQ ID NO: 15 - DNA, 23 bp, synthetic construct
  source 1..23: mol_type = other DNA
aactaccttggactttccgactg

SEQ ID NO: 16 - DNA, 19 bp, synthetic construct
  source 1..19: mol_type = other DNA
tgtggccttcaggttcagc

(19) United States
(12) Patent Application Publication
(10) Pub. No.: US 2026/0070953 A1
(43) Pub. Date: Mar. 12, 2026

(54) POMEGRANATE PGIAA9A GENE AND APPLICATION THEREOF

(71) Applicant: Institute of Horticultural Research, Anhui Academy of Agricultural Sciences, HEFEI (CN)

(72) Inventors: GAIHUA QIN, HEFEI (CN); JIYU LI, HEFEI (CN); XIN LIU, HEFEI (CN); ZHEN CAO, HEFEI (CN)

(21) Appl. No.: 19/253,224

(22) Filed: Jun. 27, 2025

(30) Foreign Application Priority Data
Jun. 29, 2024 (CN) 202410863827.9

(51) Int. Cl. C07K 14/415; C12N 15/82

(52) U.S. Cl. CPC C07K 14/415; C12N 15/8255; C12N 15/8262

(57) ABSTRACT

The present disclosure provides a pomegranate PgIAA9A gene and its encoded protein. The disclosure also provides an application of the encoded protein for regulating the growth and development of plant seeds, including negative regulation of size and lignin accumulation of the plant seed, especially negative regulation of the size and lignin accumulation of the inner seed coat of pomegranate. The pomegranate PgIAA9A gene can be used to negatively regulate the size of the inner seed coat and the lignin accumulation of pomegranate, thereby forming a basis for breeding new varieties of pomegranate with small and soft seed.

### CROSS-REFERENCE TO RELATED APPLICATIONS

This application is a U.S. Non-Provisional Application claiming priority to a Chinese Patent Application No. 202410863827.9, filed on Jun. 29, 2024, the entire contents of which are hereby incorporated fully by reference into the present disclosure.

### SEQUENCE LISTING

The Sequence Listing associated with the present application is provided in XML format, and is hereby incorporated fully by reference in its entirety into the specification. The name of the XML file containing the Sequence Listing is entitled “US93795-SeqListing_ST26XML”. The XML file was created on May 27, 2025, having a size of about 22,000 bytes, and is being submitted electronically via EFS-Web.

### FIELD

The subject matter relates to plant biotechnology, and more particularly, to a pomegranate PgIAA9A gene and an application of the same.

### BACKGROUND

Pomegranate (Punica granatum L.) is deciduous shrub or small tree belonging to the family Lythraceae, genus Punica. Pomegranate fruit is rich in nutrients and has a unique flavor, which can be eaten fresh, processed into juices, fruit wines, and other processed products. Furthermore, pomegranate may also be used as medicine. Devouring fresh pomegranate is the primary mode of consumption in China. The main edible part of pomegranate is the seed. The pomegranate seed consists of an inner seed coat, an outer seed coat, and a seed kernel. The enlarged fleshy outer seed coat is the main edible part, while the tightly structured inner seed coat is an important component of hardness of the seed. The gradual accumulation of lignin in the inner seed coat is an important factor contributing to the hardness of the pomegranate seed. The content of the lignin in the inner seed coat is positively correlated with seed hardness. Pomegranate with small and soft inner seed coats is convenient to eat and has a high edible rating, making it one of the important directions for breeding.

Auxin is a class of phytohormone that plays a crucial role in every aspect of plant growth and development. An auxin/indole-3-acetic acid (Aux/IAA) gene family is a specific class of transcription factors in plants. Encoded protein of the Aux/IAA gene can participate in auxin signaling transduction, respond quickly to small changes in auxin level, and act as a transcriptional repressor in the auxin signaling transduction system. A typical Aux/IAA gene has four structural domains. Domain I binds to an auxiliary repressor TPL/TPR to jointly inhibit transcriptional activity of a downstream auxin signaling pathway. In the presence of an auxin, a degron sequence of a GWPP-core contained in domain II binds to an auxin receptor TIR/AFB, leading to ubiquitination and degradation of an Aux/IAA protein. Point mutations in domain II can hinder the degradation of the Aux/IAA protein. Domains III and IV mediate formation of dimers between an Aux/IAA protein and an ARF or different Aux/IAA proteins, thus exerting inhibitory effects.

Currently, no related report on the Aux/IAA gene family of pomegranate has been found.

### SUMMARY

In order to solve at least one problem above and/or other potential problems in the related art, a pomegranate PgIAA9A gene and an application of the same are provided in the present disclosure.

In a first aspect, a pomegranate PgIAA9A gene is provided, wherein a nucleotide sequence of the pomegranate PgIAA9A gene is represented by SEQ ID NO:1.

In a second aspect, an encoded protein of a pomegranate PgIAA9A gene is provided, wherein an amino acid sequence of the encoded protein is represented by SEQ ID NO:3.

In a third aspect, a method for regulating seed growth and development of a plant that includes applying the pomegranate PgIAA9A gene is provided.

In a fourth aspect, a method for negatively regulating seed size and lignin accumulation of a plant that includes applying the pomegranate PgIAA9A gene is provided.

In a fifth aspect, a method for negatively regulating a size of an inner seed coat and lignin accumulation of a pomegranate that includes applying the pomegranate PgIAA9A gene is provided.

Further, applying the pomegranate PgIAA9A gene includes point-mutating the pomegranate PgIAA9A gene into a PgIAA9Am gene, where a nucleotide sequence of the

PgIAA9Am gene is represented by SEQ ID NO:2.

Further, an amino acid sequence of an encoded protein of the PgIAA9Am gene is represented by SEQ ID NO:4.

The beneficial effects of the disclosure are as follows:

The pomegranate PgIAA9A gene was cloned for the first time in the present

disclosure. The nucleotide sequence of the PgIAA9A gene is represented by SEQ ID NO.1,and the amino acid sequence of the encoded protein is represented by SEQ ID NO.3. The present disclosure has verified the biological function of the PgIAA9A gene in pomegranate for the first time through gene expression feature analysis and overexpression experiments. The PgIAA9A gene is highly expressed in the inner seed coat of pomegranate, and the expression of the PgIAA9A gene in the inner seed coat is induced by an exogenous IAA. Transgenic Arabidopsis with stable overexpression of PgIAA9A protein, which is PgIAA9Am overexpression Arabidopsis, showed smaller fruit pods, smaller seeds, shorter main roots, later development of lateral roots, and weaker plant growth potential compared to wild-type Arabidopsis (Col-0). Furthermore, lignin contents in seeds, main inflorescence stems, and rosette leaves of PgIAA9Am overexpression Arabidopsis are also reduced compared to Col-0. The content of the lignin in the inner seed coat is positively correlated with seed hardness, which indicates that less lignin accumulation can form soft inner seed coat. As such, the pomegranate PgIAA9A gene can negatively regulate the seed size and the lignin accumulation. The pomegranate PgIAA9A gene provides important genetic resource for regulating plant seed growth and development, including negative regulation of plant seed size and the lignin accumulation, especially negative regulation of the size of the inner seed coat and the lignin accumulation of pomegranate. The pomegranate PgIAA9A gene can be used for negatively regulating the size of the inner seed coat and the lignin accumulation of pomegranate, thus laying the foundation for the breeding of new varieties of pomegranate with small and soft inner seeds.

### BRIEF DESCRIPTION OF THE DRAWINGS

Implementations of the present technology will now be described, by way of example only, with reference to the attached figures.

FIG. 1 illustrates expression profiles of an Aux/IAA gene in different tissues and at different developmental stages. The different tissues include a flower, a root, a leaf, an outer seed coat, an inner seed coat, and a peel. The flower was sampled during the peak blooming period. Samples of each tissue were taken 50, 95, and 140 days after pollination (DAP) and marked as Stage 1, Stage 2, and Stage 3.

FIG. 2 is a comparison chart of relative expression levels of a PgIAA9A gene in the inner seed coat. “**” indicates P<0.01.

FIG. 3 illustrates agarose gel electrophoresis diagrams of cloned target genes of PgIAA9A and PgIAA9Am.

FIGS. 4A and 4B illustrate electrophoresis results of PCR detection of PgIAA9Am gene in transgenic plants. FIG. 4A illustrates PCR identification results of overexpression PgIAA9Am positive plants, where “WT” refers to a wild-type Arabidopsis (Col-0) plant, and “T3-OE” refers to a T3 generation overexpression PgIAA9Am

Arabidopsis plant. FIG. 4B illustrates semi-quantitative PCR analysis results of PgIAA9Am overexpression plants, where “WT” refers to a wild-type Arabidopsis (Col-0) plant, “T3-OE-1” to “T3-OE-8” refer to T3 generation overexpression PgIAA9Am Arabidopsis plants, and “AtActin” refers to an internal reference gene of an Arabidopsis.

FIGS. 5A-5G illustrate plant phenotypes as well as growth and development related traits of overexpression PgIAA9Am Arabidopsis plants. FIG. 5A illustrates phenotypes of seedlings that are grown on a 1/2 Multiple Sclerosis (MS) solid medium for 15 days. FIG. 5B illustrates phenotypes of plants that are grown in a cultivation soil for 30 days. FIG. 5C illustrates the phenotypes of the plants that are grown in the cultivation soil for 45 days. FIG. 5D illustrates relative numbers of rosette leaves of the plants that are grown in the cultivation soil for 45 days. FIG. 5E illustrates rosette leaf diameters of the plants that are grown in the cultivation soil for 45 days. FIG. 5F illustrates fresh weights of above-ground parts of the plants that are grown in the cultivation soil for 45 days. FIG. 5G illustrates dry weights of the above-ground parts of the plants that are grown in cultivation soil for 45 days where “WT” refers to a wild-type Arabidopsis (Col-0) plant, “OE #1” and “OE #2” refer to T3 generation Arabidopsis plants overexpression PgIAA9Am, and “**” indicates p<0.01.

FIGS. 6A-6F illustrate phenotypes of fruit pods and mature seeds of PgIAA9Am overexpression Arabidopsis. FIG. 6A illustrates the phenotypes of the fruit pods that are grown in a cultivation soil for 45 days. FIG. 6B illustrates mature grain phenotypes (that are grown in a cultivation soil for 60 days). FIG. 6C illustrates 1000-grain weights of mature seeds (that are grown in a cultivation soil for 60 days. FIG. 6D illustrates longitudinal diameters of the mature seeds (that are grown in the cultivation soil for 60 days). FIG. 6E illustrates relative diameters of the mature seeds (that are grown in the cultivation soil for 60 days). FIG. 6F illustrates lengths of the fruit pods (that are grown in the cultivation soil for 45 days), where “WT” refers to a wild-type Arabidopsis (Col-0) plant, “OE #1” and “OE #2” refer to T3 generation overexpression PgIAA9Am Arabidopsis plants, “*” indicates p<0.05, and “**” indicates p <0.01.

FIG. 7A illustrates lignin contents in mature seeds, FIG. 7B illustrates main inflorescence stems, and FIG. 7C illustrates rosette leaves of overexpression PgIAA9Am Arabidopsis plants, where “WT” refers to a wild-type Arabidopsis (Col-0) plant, “OE #1” and “OE #2” refer to T3 generation overexpression PgIAA9Am Arabidopsis plants, and “**” indicates p<0.01.

### DETAILED DESCRIPTION

It will be appreciated that for simplicity and clarity of illustration, where appropriate, reference numerals have been repeated among the different FIGS. to indicate corresponding or analogous components. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

1. Analysis of the expression pattern of pomegranate PgIAA9A gene.

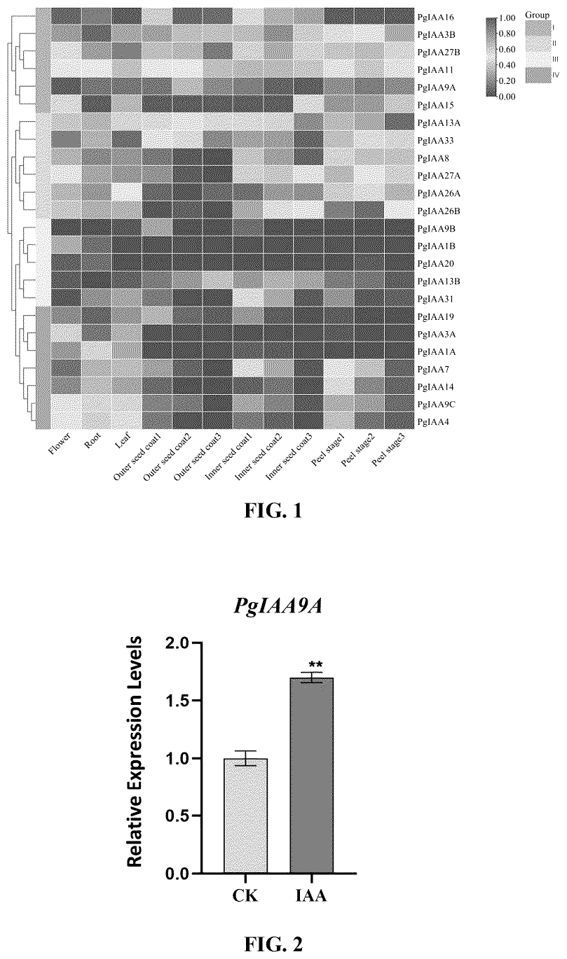

Referring to FIG. 1, RNA-seq data for different tissues of the pomegranate cultivar “Dabenzi”, including flowers, roots, leaves, peels, and inner and outer seed coats were analyzed, which revealed that the PgIAA9A gene is expressed in any one of these tissues mentioned above. At various developmental stages of the inner and outer seed coats, the expression levels of the PgIAA9A gene were different, and the PgIAA9A gene shows significantly a higher expression level in the inner seed coat than in the outer seed coat.

At a key period of seed hardness formation (60 days after pollination), pomegranate fruits with consistent growth and good development were selected. Cotton soaked in 10 μmol/L of IAA (indoleacetic acid) was wrapped around fruit stalks, then the cotton was covered by a tinfoil tightly to keep the cotton in place. A control (CK) fruit stalk was treated with another cotton soaked in distilled water. After a 12-hour treatment, the inner seed coat tissues from both the CK group and the IAA treatment group were collected, flash-frozen in liquid nitrogen, and stored at −80° C. for later use or directly used in subsequent experiments.

A polysaccharide polyphenol plant total RNA extraction kit (TIANGEN Biotech (Beijing) Co., Ltd) was used to extract an RNA from the pomegranate inner seed coats of the CK group and the IAA treatment group. Reverse transcription was performed using a reverse transcription kit from Bioer Technology (Beijing) Co., Ltd. to obtain a cDNA. Using the cDNA of the inner seed coat as template, a qRT-PCR amplification was performed using an AceQ qPCR SYBR Green Master mix reagent. The mix reagent includes primers PgActin7-F and PgActin7-R, PgIAA9AqRT-PCR-F and PgIAA9AqRT-PCR-R. A relative expression level of the PgIAA9A gene in the pomegranate inner seed coat, after IAA treatment, was calculated using the 2^{ΔΔCt} method. As shown in FIG. 2, the expression level of the PgIAA9A gene in the pomegranate inner seed coat increased significantly after the exogenous IAA treatment, indicating that exogenous IAA could induce the expression of a PgIAA9A gene in a pomegranate inner seed coat.

2. Cloning of PgIAA9A gene.

A polysaccharide polyphenol plant total RNA extraction kit (TIANGEN Biotech (Beijing) Co., Ltd) was used to extract an RNA from the pomegranate inner seed coats of “Dabenzi”. Reverse transcription was performed using a reverse transcription kit from Bioer Technology (Beijing) Co., Ltd. to obtain a cDNA. After a 5-fold dilution of the cDNA with double-distilled water (ddH₂O), a polymerase chain reaction (PCR) amplification of the target gene PgIAA9A was performed. The amplified sequence is represented by SEQ ID NO.1, and an amino acid sequence of a PgIAA9A encoded protein is represented by SEQ ID NO.3. The amplification system includes PgIAA9A-F used as a forward primer, PgIAA9A-R used as a reverse primer, and a KOD high-fidelity enzyme (e.g., Phanta MaxSuper-Fidelity DNAPolymerase).

Due to the presence of large amounts of auxin during seed development, it was difficult to obtain stably expressed PgIAA9A protein after overexpressing PgIAA9A in Arabidopsis. Through point mutation, the first proline in the amino acid sequence GWPP encoded by the codon in domain II of PgIAA9A was mutated to serine, that is GWSP. Thus, the resulting gene after point mutation was PgIAA9Am, which could stably obtain the expressed PgIAA9A protein.

The cloning method of PgIAA9Am was as follows: a PCR was performed using PgIAA9A-F and PgIAA9Am-R to obtain the first PCR product, then another PCR was performed using PgIAA9Am-F and PgIAA9A-R to obtain the second PCR product. The first and second PCR products were adjusted to the same concentration, and mixed in equal volume, then anther PCR was performed using PgIAA9A-F and PgIAA9A-R to obtain a third product. A nucleotide sequence of the third product is represented by SEQ ID NO.2,which is PgIAA9Am. An amino acid sequence of a protein encoded by PgIAA9Am is represented by SEQ ID NO.4.

The PCR reaction system is shown in Table 1 below:

TABLE 1

| Reagent | Usage |
|---|---|
| cDNA | 1 μL |
| Forward primer | 1 μL |
| Reverse primer | 1 μL |
| 10× Buffer | 5 μL |
| Phanta MaxSuper-Fidelity DNAPolymerase | 1 μL |
| 2 mM dNTP Mixture | 5 μL |
| 25 mM MgSO₄ | 3 μL |
| ddH₂O | 33 μL |
| Total | 50 μL |

The PCR reaction program includes: pre-denaturation at 95° C. for 3 min.; denaturation at 95° C. for 30 seconds, annealing at 59° C. for 30 seconds, extension at 72° C. for 1 min./kb, 33 cycles; extension at 72° C. for 10 min.; and the PCR product was stored at 4° C.

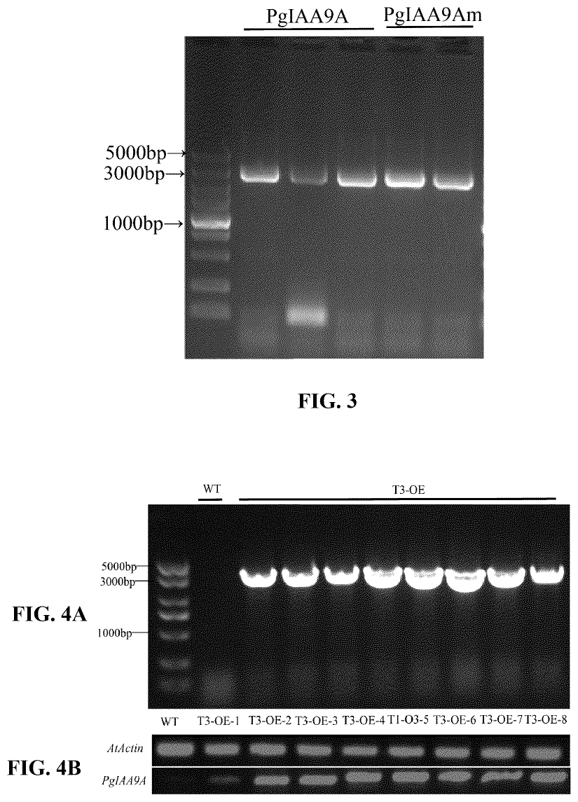

The PCR products were detected by 1% agarose gel electrophoresis. Under UV conditions, an observation was made by a gel imaging system, and the results showed that single bright bands of approximately 2600bp have been amplified (as shown in FIG. 3). The gels were cut under UV irradiation, and target fragments were recovered using a DNA gel recovery kit from Beijing Jinsha Biotechnology Co., Ltd., according to an instruction manual in the kit. Then the recovered fragments were sequenced also according to the instruction manual, and the sequences are represented by SEQ ID NO.1 and SEQ ID NO.2, respectively.

3. Construction PgIAA9Am overexpression vector PgIAA9Am-pCAMBIA1300GFP.

An expression vector pCAMBIA1300GFP was digested with restriction enzymes, BamHI and KpnI.

An enzymatic digestion system is shown in Table 2 below:

TABLE 2

| Reagent | Usage |
|---|---|
| Q Cut KpnI | 2 μL |
| Q Cut BamHI | 2 μL |
| Q Cut Buffer | 5 μL |
| pCAMBIA1300GFP plasmid | 10 μL |
| ddH₂O | 31 μL |
| Total | 50 μL |

The reaction mixture was mixed and reacted at

The system is shown in Table 3 below:

TABLE 3

| Reagent | Usage | |
|---|---|---|
| PCR product fragments | 100 | ng |
| Vector fragments | 150 | ng |
| DNA ligase | 10 | μL |
| ddH₂O | 31 | μL |

The reaction mixture was mixed and reacted at 50° C. for 15 min. to obtain a recombination product. After the reaction, the recombination product was cooled on ice for several seconds, then the recombination product could be stored at −20° C. for later use or directly used for transformation. The recombination product was transformed into Escherichia coli DH5α, then screened on a Luria-Bertani (LB) solid medium containing 50 mg/L kanamycin. Single colonies from the transformation plate were picked as templates for PCR identification using the primers required for cloning the PgIAA9Am mentioned above. To increase the accuracy of identification results, PgIAA9A-F could be replaced with PgIAA9A-1300GFP-F, or PgIAA9A-R could be replaced with PgIAA9A-1300GFP-R.

In an embodiment, a preparation method of LB solid medium containing 50 mg/L of kanamycin was as follows: 5 g of tryptone, 2.5 g of yeast extract, and 5 g of sodium chloride were mixed to form a mixture. Then ddH₂O was added to bring the mixture to a volume of 1 L, the pH of the mixture was adjusted to about 7.0, and then 10 g of agar powder was added in the mixture. The final mixture was autoclaved at 121° C. for 20 min. After sterilization, the kanamycin was added.

The reaction program includes: pre-denaturation at 95° C. for 3 min.; denaturation at 95° C. for 30 seconds, annealing at 59° C. for 30 seconds, extension at 68° C. for 2 min., 33 cycles; final extension at 72°° C. for 10 min.; and the PCR product was stored at 4° C.

The colony PCR reaction system is shown in Table 4 below:

TABLE 4

| Reagent | Usage |
|---|---|
| Forward primer | 1 μL |
| Reverse primer | 1 μL |
| MIX containing Taq enzyme | 10 μL |
| Template | Single colony |
| ddH₂O | Up to 50 μL |

The PCR products were detected by 1% agarose gel electrophoresis. The positive clone identification results were observed under UV conditions using the gel imaging system. The recombinant plasmid (PgIAA9Am-pCAMBIA1300GFP) from the positive single clone was extracted for sequencing.

4. Transformation of the PglAA9Am overexpression vector PglAA9Am-pCAMBIA1300GFP into Agrobacterium.

After confirming the correct sequence of the recombinant plasmid (PgIAA9Am-pCAMBIA1300GFP), it was transformed into Agrobacterium competent cells (GV3101) by a chemical transformation method. In an embodiment, the method included the following steps: 1 ug of recombinant plasmid was added into 50 uL of Agrobacterium competent cells and mixed gently to obtain a mixture. The mixture was iced for 30 min., and flash frozen in liquid nitrogen for 5 min., then thawed at a 37° C. water bath for 5 min. 700 μL of YEP liquid medium was added into the mixture. Then the mixture was shaken at 200 rpm, at 30° C., for 3-4 hours, and centrifuged at 5000 rpm for 5 min to obtain a final mixture. 100 μL˜200 μL of the final mixture was taken to spread on a YEP solid medium containing 50 mg/L of kanamycin and 50 mg/L of rifampicin for screening. Single colonies were PCR amplified (same as PCR identification in section 3). The single colonies all showed a single bright band consistent with the size of the target gene, indicating that the PgIAA9Am gene overexpression vector PgIAA9Am-pCAMBIA1300GFP was successfully transformed into Agrobacterium competent cells.

In an embodiment, a preparation method of the 700 μL of YEP liquid medium was as follows: 2.5 g of yeast extract, 5 g of peptone, and 5 g of sodium chloride were mixed to obtain a mixture. Then ddH₂O was added to bring the mixture to a volume of 1 L, and the pH of the mixture was adjusted to about 7.0. Finally, the mixture was autoclaved at 121° C. for 20 min.

In an embodiment, a preparation method of the YEP solid medium containing the 50 mg/L of kanamycin and the 50 mg/L of rifampicin was as follows: 2.5 g of yeast extract, 5 g of peptone, and 5 g of sodium chloride were mixed to obtain a mixture, ddH₂O was added to bring the mixture to a volume of 1 L, the pH of the mixture was adjusted to about 7.0, and 10 g of agar powder was added into the mixture. Then the mixture was autoclaved at 121° C. for 20 min. After sterilization, the kanamycin and the rifampicin were added into the mixture.

5. Arabidopsis infection and transgenic plant screening.

A positive single colony of Agrobacterium was picked and added into 5 mL of a yeast extract peptone (YEP) liquid medium containing 50 mg/L of kanamycin and 50 mg/L of rifampicin, then cultured in a 28° C. incubator with shaking at 200 rpm for 16 hours to obtain a first Agrobacterium suspension. 50 uL of the first Agrobacterium suspension was took and added into 50 mL of YEP liquid medium containing 50 mg/L of kanamycin and 50 mg/L of rifampicin, then cultured in a 28° C. incubator with shaking at 220 rpm for about 16 hours to obtain a second Agrobacterium suspension. An absorbance value measured at 600 nm (OD600 value) of the second Agrobacterium suspension is 0.8 to 1.0.

The second Agrobacterium suspension was centrifuged at 5000 rpm for 6-8 min., supernatant was removed, then Agrobacterium tumefaciens were collected. Resuspend the

Agrobacterium tumefaciens in 1/2 MS liquid medium containing 50 mg/mL of sucrose to obtain a resuspended Agrobacterium tumefaciens. The OD600 value of the resuspended Agrobacterium tumefaciens is about 0.8. Then 0.2 uL/mL of Silwetl-77 was added into the resuspended Agrobacterium tumefaciens to obtain an Agrobacterium infection solution. When infecting Arabidopsis, a wild-type Arabidopsis that is in the flowering stage and growing robustly was selected. Mature fruit pods and open inflorescences of the Arabidopsis were removed, and unopened inflorescences were left. The Arabidopsis absorbed enough water before infection, then the inflorescences of the Arabidopsis were soaked in the Agrobacterium infection solution for 35-60 seconds. Then the Arabidopsis was first cultivated in a light incubator (at 21±1° C. and a humidity of 70%-80%) in darkness for 1 day, then switch to normal light (which is cultivated in light for 16 hours and in darkness for 8 hours). After the first infection, the inflorescences were performed a second infection one week later according to the method mentioned above. Before the second infection, the mature fruit pods and open inflorescences did not need to be removed.

When the fruit pods were mature, the T0 generation seeds were collected. Then the seeds were placed in a 1.5 mL centrifuge tube for storage, and a color-changing silica gel desiccant was added into the tube. The T0 generation seeds were sowed on a 1/2 MS solid medium containing 200 μg/mL of carbenicillin and 30 μg/mL of hygromycin. Then the seeds in the solid medium were cultivated in a light incubator with normal light conditions (at 21±1° C., a humidity of 70%-80%, and a light cycle of 16 hours of light and 8 hours of darkness). The seedlings of Arabidopsis with successful overexpression of PgIAA9Am, during grain germination in the T0 generation, can maintain normal growth of leaves and roots. However, the Arabidopsis seedlings with failed overexpression of PgIAA9Am gradually turn yellow in cotyledons and their roots no longer elongate, unable to continue growing, and eventually die.

Subsequently, seeds from individual plants of T1 and T2 generations were collected and sowed on a 1/2 MS solid medium containing 30 ug/mL of hygromycin. The seeds on the solid medium were cultivated in a light incubator (at 21±1° C., with a humidity of 70%-80%, and light for 16 hours and darkness for 8 hours) and then homozygotes were screened out.

DNA was extracted from the leaf tissue of T3 generation Arabidopsis using the CTAB method, and a full-length PCR detection of PgIAA9Am was performed. In order to increase the accuracy of the identification results, in the primers required for the PgIAA9Am cloning, PgIAA9A-F can be replaced with PgIAA9A-1300GFP-F, or PgIAA9A-R can be replaced with PgIAA9A-1300GFP-R. A band consistent with the size of the target gene fragment was detected in multiple PgIAA9Am overexpression lines (as shown in FIG. 4A), indicating that PgIAA9Am was successfully expressed at the DNA level of Arabidopsis. RNA was extracted from the rosette leaf tissue of the overexpression PgIAA9Am

Arabidopsis lines in the T3 generation. Then a semi-quantitative PCR analysis was performed using the reverse-transcribed cDNA as a template, with primers PgActin7-F and PgActin7-R, AtActin2-F and AtActin2-R. The results as shown in FIG. 4B, which indicated that the PgIAA9Am overexpression lines showed clear bands, while the wild-type plants showed no bands.

In an embodiment, a preparation method of the 5 mL of YEP liquid medium containing 50 mg/L of kanamycin and 50 mg/L of rifampicin was as follows: firstly, 2.5 g of yeast extract, 5 g of peptone, and 5 g of sodium chloride were mixed to obtain a mixture. Secondly, ddH2O was added to bring the mixture to a volume of 1 L, and the pH of the mixture was adjusted to about 7.0. Thirdly, the mixture was autoclaved at 121° C. for 20min. Finally, the kanamycin and the rifampicin were added in the mixture after sterilization.

In an embodiment, a preparation method of the 1/2 MS liquid medium containing 50 mg/mL of sucrose was as follows: firstly, MS powder of 2.5 g/L, sucrose of 50 g/L, and 2-morpholinoethanesulfonic acid (MES) at 0.5 g/L were mixed to obtain a mixture. Secondly, the pH of the mixture was adjusted to about 5.8. Finally, the mixture was autoclaved at 121° C. for 20 min.

In an embodiment, a preparation method of the 1/2 MS solid medium containing 200 ug/mL of carbenicillin and 30 ug/mL of hygromycin is as follows: firstly, 2.5 g/L of MS powder, 20 g/L of sucrose, and 0.5 g/L of MES were mixed to obtain a mixture. Secondly, the pH of the mixture was adjusted to around 5.8. Thirdly, 8 g/L of agar powder was added in the mixture. Fourthly, the mixture was placed in a high-pressure steam sterilization pot to sterilized at 121° C. for 20 minutes. After sterilization, the carbenicillin and the hygromycin were added into the mixture.

In an embodiment, a preparation method of the 1/2 MS solid medium containing 30 ug/mL of hygromycin is as follows: MS powder of 2.5 g/L, sucrose of 20 g/L, and MES of 0.5 g/L were mixed to obtain a mixture. Secondly, the pH of mixture was adjusted to around 5.8, and then agar powder at 8 g/L was added. Thirdly, the mixture was placed in a high-pressure steam sterilization pot to sterilized at 121° C. for 20 minutes.

After sterilization, the hygromycin was added in the mixture.

6. Phenotype identification of PgIAA9Am gene overexpression plants.

Following the above Arabidopsis infection and transgenic plant screening, two homozygous lines with different PgIAA9Am expression levels were selected, namely

OE #1 and OE #2 (where OE #1 had a higher expression level). To investigate growth and development performance of the two homozygous lines, with a wild-type Arabidopsis plant as control, which is named WT. Specific operations were as follows: normal T3 generation plants screened on 1/2 MS solid medium containing 30 ug/mL of hygromycin were transplanted to plug trays containing a cultivation soil (a volume ratio of vermiculite: the nutrient soil=3:1) after developing four true leaves. Growth conditions remained unchanged (at 21±1° C., a humidity of 70%-80%, and 16 hours of light and 8 hours of darkness). Plant phenotypes were observed and photographed after 30 days of growth in the cultivation soil. Then plant phenotypes were observed and photographed after 45 days of growth in the cultivation soil. Plant height, above-ground biomass or part (including fresh weight and dry weight of the above-ground mass/part), the number of rosette leaves, and rosette leaf diameters were observed and measured. Their siliques were observed, photographed and measured. After 60 days of growth in the cultivation soil, thousand- grain weight, longitudinal diameter, and transverse diameter of mature seeds were measured. For plants requiring root observation, T2 generation seeds were spotted on a 1/2 MS solid medium (with a preparation method including: mixing MS powder of 2.5 g/L, sucrose of 20 g/L, and MES of 0.5 g/L; [,] adjusting the pH to about 5.8; [,] then adding 8 g/L of agar powder; and autoclaving at 121° C. for 20 min.) and placed vertically (at 21±1° C., a humidity of 70%-80%, 16 hours of light and 8 hours of darkness), then photographed daily.

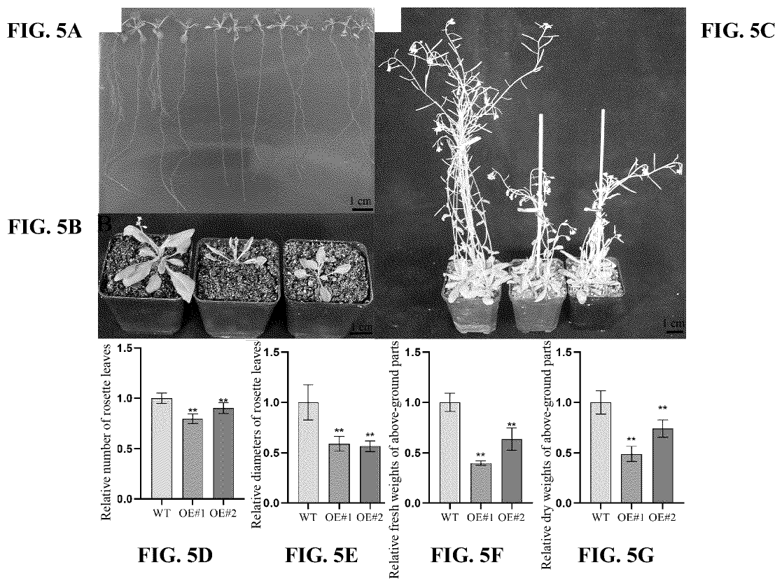

Compared with the wild-type plants, PgIAA9Am overexpression plants showed shorter primary roots and slower lateral root growth after 15 days of growth on 1/2 MS solid medium (as shown in FIG. 5A). After 30 days of growth in soil, they showed a slower growth rate and curled rosette leaves (as shown in FIG. 5B). Observation and measurement of plant height, above-ground biomass, the number of rosette leaves and rosette leaf diameter of plants that are grown in soil for 45 days showed that PgIAA9Am overexpression plants had significantly lower plant height and above-ground biomass than wild-type plants (as shown in FIGS. 5C, 5F, and 5G), and significantly fewer rosette leaves (as shown in FIG. 5D) and smaller rosette leaf diameter (as shown in FIG. 5E). Phenotype after 45 days of growth in soil is shown in FIG. 5C. These characteristics indicate that root development and vegetative growth of PgIAA9Am overexpression plants were significantly inhibited.

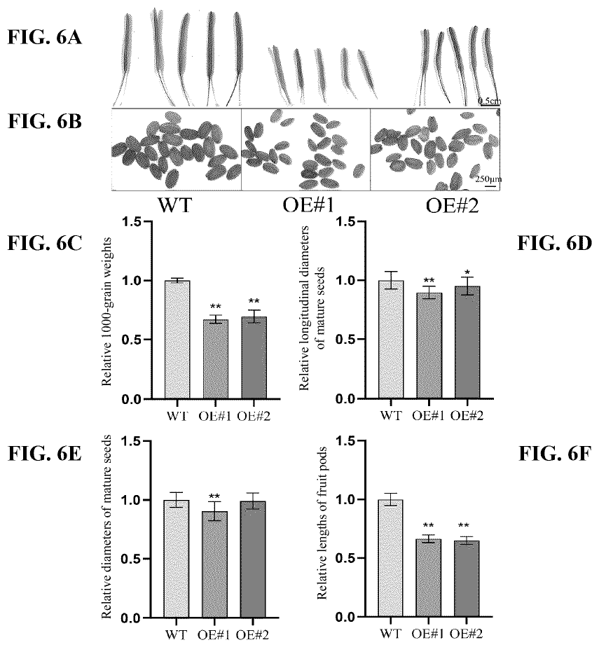

Compared with wild-type siliques and mature seeds, PgIAA9Am overexpression also had significant effects on siliques and mature seeds (as shown in FIG. 6A and 6B), with significantly reduced silique length (as shown in FIG. 6F), thousand- grain weight (as shown in FIG. 6C), longitudinal diameter (as shown in FIG. 6D), and relative diameter (as shown in FIG. 6E) of mature seeds in PgIAA9Am overexpression plants. This indicates that PgIAA9Am overexpression has a significant inhibitory effect on seed growth and development.

7. Determination of lignin content and observation of lignin accumulation in PgIAA9Am gene overexpression plants.

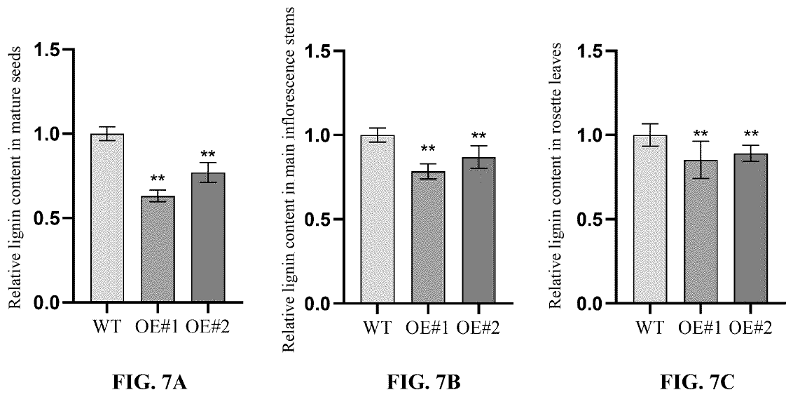

Following the above phenotype identification of PgIAA9Am gene overexpression plants, lignin contents were measured in rosette leaves of the plants that are grown in soil after 15 days. Lignin contents were measured in the main inflorescence stems of the plants that are grown in soil after 45 days. Lignin contents were measured in mature seeds of the plants that are grown in soil after 60 days. Lignin content was measured by a BC4200 kit from Beijing Solarbio Science & Technology Co., Ltd. Results of the lignin contents determination in PgIAA9Am overexpression plants showed that both overexpression lines (OE #1 and OE #2) had significantly lower lignin content in mature seeds, main inflorescence stems, and rosette leaves compared to wild-type plants (as shown in FIG. 7). Compared to the wild-type plants, the lignin contents in mature seeds of the two overexpression lines decreased by 37% and 23%, respectively, the lignin contents in main inflorescence stems of the two overexpression lines decreased by 21% and 13%, respectively, and the lignin contents in rosette leaves of the two overexpression lines decreased by 14% and 10%, respectively.

8. The primers used in the above embodiments are shown in Table 5.

TABLE 5

| Primer | |
|---|---|
| sequence | 5′to3′ |
| PgIAA9A-F | ATGTCTCCACCGCTCCTGGGT |
| PgIAA9A-R | TCAGTTTCTTGCCTTGGATTTCT |
| PgIAA9Am-F | CAGGTCATTGGTTGGTCTCCAGTGAGATCCTTCAG |
| PgIAA9Am-R | CTGAAGGATCTCACTGGAGACCAACCAATGACCTG |
| PgIAA9A- | CACGGGGGACGAGCTCGGTACCATGTCTCCACCGC |
| 1300GFP-F | TCCTGGGT |
| PgIAA9A- | CCATGTCGACTCTAGAGGATCCTCAGTTTCTTGCC |
| 1300GFP-R | TTGGATTTCT |
| PgActin7-F | TATTGTTGGTCGTCCCAGGC |
| PgActin7-R | CCTCTCTTCGATTGGGCCTC |
| AtActin2-F | GGTAACATTGTGCTCAGTGGTGG |
| AtActin2-R | AACGACCTTAATCTTCATGCTGC |
| PgIAA9AqRT- | AACTACCTTGGACTTTCCGACTG |
| PCR-F | |
| PgIAA9AqRT- | TGTGGCCTTCAGGTTCAGC |
| PCR-R | |

Even though information and advantages of the present embodiments have been set forth in the foregoing description, together with details of the structures and functions of the present embodiments, the disclosure is illustrative only. Changes may be made in detail, especially in matters of shape, size, and arrangement of parts within the principles of the present exemplary embodiments, to the full extent indicated by the plain meaning of the terms in which the appended claims are expressed.

SEQUENCE LISTING
SEQ ID NO: 1:
a nucleotide sequence of the pomegranate PgIAA9A
gene
ATGTCTCCACCGCTCCTGGGTGCGGAGGAAGGAGGAACAAGCGATGTCAC
CAAAGTTGCTGGTTCTCCCTCCTCAAAGGACTCCCCTGATGGCCTTGGCT
TGGGCCTGAAAGAGAGGAACTACCTTGGACTTTCCGACTGTTCATCGGTT
GACAGTTCAACGGTGTCGGGCCAGTCAGATCCTGAGGCCCAGAACAACAA
GCTGAACCTGAAGGCCACAGAGCTCAGGCTTGGGCTTCCAGGATCTCAGT
CCCCGGAAAGAGATCTTGATGAGAAGCCTCTGTTCCCTCTCCTTCCCTCA
AAGGATGGGATCAAGACTGTCGTCTCAGGCAATAAGAGGGGTTTCTCTGA
TGCAATTGATGGGTTCTCGGATAAGAAGTGGATGTTTCACTCTGCCGGGA
CTGATGAGGGCACACAGCCATTGGGACAGGGTAAGTTTCCTGCTAATGCT
GGCCCTAAGCAGCCAGCTACCAAAGAGGTGCCGGCAAAGCCCCTGCCTGA
ACGAGCAAATGGGATTAACCCGAGCAGAGCTGCGAACAATGCACCAGCTG
CCAAGGCCCAGGTCATTGGTTGGCCTCCAGTGAGATCCTTCAGAAAGAAC
ACTTTGGCGACTACTTCTAAGGATAATGATGAAGTAGATGGAAAACCTGG
TCCTGGTGCTCTCTTCATCAAGGTGAGCATGGATGGTGCTCCATATTTGC
GGAAGGTGGACCTAAGGAACTACTCCAAGTACTATGAACTCTCCGTTGCT
CTCGAGAAGATGTTCAGCGGTTTTACCATAGGGCAATGTGGAACTAATGG
AACTGGTGGAAGGGAATCGATGAGCGAGAGCAAGCTGAAGGATCTTCTGC
ATGGGTCAGACTATGTTCTCACATACGAGGACAAAGATGGTGATTGGATG
CTAGTTGGAGATGTCCCATGGGAATGCCCCCTTCCCCCCCTCTTGGCCGC
CTCTGCGCTCTCAGGAATCGAGATCTCGGATCGTCGCGCCTCTGCTTCAT
CGATTCAGCTCGCCGCGATCTCTCAGTTCTCTTTGGTAAGATTTCAAGAG
ATTGGCTCAAGAATGCGAGGATACGAAGATGATGAGTACGAGGATTATGA
TAATTATGAGGAGGAAGGGTACGAGCAAGAGGAAGGCGGTGATGAAGAAG
ATGAATATGAAGAGGAAGAAGAAGAGCCAAAGCCCACCAAGGAGGAATTG
GAATATTTGGAGTTGAGGCAGAAACTGAAGGAATCTATCAGGAAGCGGAT
GAAGAAGGCAGCTTCTGGTTCAGAAGAGATAAAGAAAGACAATTATGGCT
CTTTCTTTGGCCCTTCTAAACCTGTCATTGCACAGCGAGTGATTCAAGAG
AGCAAGTCATTGTTGGAGAATCAACATTTGGCTGCCCGATTGTTGAAGTC
CCATCAGAATGGTAATAAGAGTTCTTCTTCAAGACCTGATGGTTCCAAGC
CTGGAGTGCGTCCCCAAGCTCCCAGAGTAAGCCAGCTGAAACAGAGAGTT
CAGAAAATAAAAGATACAAGGGACTACTCTTTTCTATTATCTGATGATGC
TGAGCTCCCTGCCTCGCCCAAACAACCTCCACCACGAAGTATCCCTGCTC
CCAGTTTGGATGCTCGCTCAGCTAAATTATCGTCTCAGAGCAGACTTAAC
CCAGGGAGCAATAGCAGACATGTAAATGGTGGTCGTGAAGAAAGAAGACC
AGCTTCATCTAATCGTCACAGTAACCCTAGACCAGGCTCCTCTAATGGTC
ACAGTAATAATAGACCTGGATCTGCTAATGGTTATAGCAACACCAGACCA
GGCTCTGCTAATGGTCACAGTAACCTCAGACCAGGCTCCGCTAATGTTCA
CAGTAACCTCAGACCAGGCTCCGCTAATGGTCACAGTAACCTCAGACCAG
GCTCCGCCAATGGTCACAGTAACCTCAGACCAGGCTCCGCCAATGGTCAC
AGTAATCTTGGAGCCGGACCAAATAAGTTGTCTTCTGCTAGTAAGCCTCA
GATGGCAGCCAATAGGAAGCAGCTCGGTAGCAACAGTGGGAATGGGCCTG
GCCGGCCTGCAGTTTCGAATGGGTTGCAACCAAAGAAGCCAATTCCACCC
CCACAGAGATCTTCTCAAGGTTTGAAAAGCTCTGTTCCTGCTGGGGTGAA
ACCGCCAGCCACAAAGATGCAGTCCTCTGCTTCGAAGTACCAGGTGGAAC
CAAGAAGAAATGTGCAGCAACCAAACAAGGGCAAAATTTTATCTAAACAT
TCGGTTGCTTCGACTAGACCTCAGGTTCAAGCTCAGATGAGCAAACCTCA
GAAGCAAATCCCCTCTCATAACAGACTGCAAGATCAGAGGCCCAAGAAAA
GGCCCAGCAGACCATTCCCTGAAGACGACGATGACAATGATGCGGCCATC
AGTATGATCAGAAAAATGTTTGGATACAATCCGCAAAAGTTTGCTGGTCG
TGATGAAGACGACAGTGATATGGAAGCAAACTTTGACGACATCATGAGGG
AAGAAAAGAGGAGTGCGAGAATCGCCCAAAAGGAGGACGAGGAACAGCTG
CGCTTGATAGAGGAGGAGGAAAGGCGCGAACGCGAACGGATAAGAAAAAA
GCGGAAGATGGGATAA
SEQ ID NO: 2:
a nucleotide sequence of the PgIAA9Am gene
ATGTCTCCACCGCTCCTGGGTGCGGAGGAAGGAGGAACAAGCGATGTCAC
CAAAGTTGCTGGTTCTCCCTCCTCAAAGGACTCCCCTGATGGCCTTGGCT
TGGGCCTGAAAGAGAGGAACTACCTTGGACTTTCCGACTGTTCATCGGTT
GACAGTTCAACGGTGTCGGGCCAGTCAGATCCTGAGGCCCAGAACAACAA
GCTGAACCTGAAGGCCACAGAGCTCAGGCTTGGGCTTCCAGGATCTCAGT
CCCCGGAAAGAGATCTTGATGAGAAGCCTCTGTTCCCTCTCCTTCCCTCA
AAGGATGGGATCAAGACTGTCGTCTCAGGCAATAAGAGGGGTTTCTCTGA
TGCAATTGATGGGTTCTCGGATAAGAAGTGGATGTTTCACTCTGCCGGGA
CTGATGAGGGCACACAGCCATTGGGACAGGGTAAGTTTCCTGCTAATGCT
GGCCCTAAGCAGCCAGCTACCAAAGAGGTGCCGGCAAAGCCCCTGCCTGA
ACGAGCAAATGGGATTAACCCGAGCAGAGCTGCGAACAATGCACCAGCTG
CCAAGGCCCAGGTCATTGGTTGGTCTCCAGTGAGATCCTTCAGAAAGAAC
ACTTTGGCGACTACTTCTAAGGATAATGATGAAGTAGATGGAAAACCTGG
TCCTGGTGCTCTCTTCATCAAGGTGAGCATGGATGGTGCTCCATATTTGC
GGAAGGTGGACCTAAGGAACTACTCCAAGTACTATGAACTCTCCGTTGCT
CTCGAGAAGATGTTCAGCGGTTTTACCATAGGGCAATGTGGAACTAATGG
AACTGGTGGAAGGGAATCGATGAGCGAGAGCAAGCTGAAGGATCTTCTGC
ATGGGTCAGACTATGTTCTCACATACGAGGACAAAGATGGTGATTGGATG
CTAGTTGGAGATGTCCCATGGGAATGCCCCCTTCCCCCCCTCTTGGCCGC
CTCTGCGCTCTCAGGAATCGAGATCTCGGATCGTCGCGCCTCTGCTTCAT
CGATTCAGCTCGCCGCGATCTCTCAGTTCTCTTTGGTAAGATTTCAAGAG
ATTGGCTCAAGAATGCGAGGATACGAAGATGATGAGTACGAGGATTATGA
TAATTATGAGGAGGAAGGGTACGAGCAAGAGGAAGGCGGTGATGAAGAAG
ATGAATATGAAGAGGAAGAAGAAGAGCCAAAGCCCACCAAGGAGGAATTG
GAATATTTGGAGTTGAGGCAGAAACTGAAGGAATCTATCAGGAAGCGGAT
GAAGAAGGCAGCTTCTGGTTCAGAAGAGATAAAGAAAGACAATTATGGCT
CTTTCTTTGGCCCTTCTAAACCTGTCATTGCACAGCGAGTGATTCAAGAG
AGCAAGTCATTGTTGGAGAATCAACATTTGGCTGCCCGATTGTTGAAGTC
CCATCAGAATGGTAATAAGAGTTCTTCTTCAAGACCTGATGGTTCCAAGC
CTGGAGTGCGTCCCCAAGCTCCCAGAGTAAGCCAGCTGAAACAGAGAGTT
CAGAAAATAAAAGATACAAGGGACTACTCTTTTCTATTATCTGATGATGC
TGAGCTCCCTGCCTCGCCCAAACAACCTCCACCACGAAGTATCCCTGCTC
CCAGTTTGGATGCTCGCTCAGCTAAATTATCGTCTCAGAGCAGACTTAAC
CCAGGGAGCAATAGCAGACATGTAAATGGTGGTCGTGAAGAAAGAAGACC
AGCTTCATCTAATCGTCACAGTAACCCTAGACCAGGCTCCTCTAATGGTC
ACAGTAATAATAGACCTGGATCTGCTAATGGTTATAGCAACACCAGACCA
GGCTCTGCTAATGGTCACAGTAACCTCAGACCAGGCTCCGCTAATGTTCA
CAGTAACCTCAGACCAGGCTCCGCTAATGGTCACAGTAACCTCAGACCAG
GCTCCGCCAATGGTCACAGTAACCTCAGACCAGGCTCCGCCAATGGTCAC
AGTAATCTTGGAGCCGGACCAAATAAGTTGTCTTCTGCTAGTAAGCCTCA
GATGGCAGCCAATAGGAAGCAGCTCGGTAGCAACAGTGGGAATGGGCCTG
GCCGGCCTGCAGTTTCGAATGGGTTGCAACCAAAGAAGCCAATTCCACCC
CCACAGAGATCTTCTCAAGGTTTGAAAAGCTCTGTTCCTGCTGGGGTGAA
ACCGCCAGCCACAAAGATGCAGTCCTCTGCTTCGAAGTACCAGGTGGAAC
CAAGAAGAAATGTGCAGCAACCAAACAAGGGCAAAATTTTATCTAAACAT
TCGGTTGCTTCGACTAGACCTCAGGTTCAAGCTCAGATGAGCAAACCTCA
GAAGCAAATCCCCTCTCATAACAGACTGCAAGATCAGAGGCCCAAGAAAA
GGCCCAGCAGACCATTCCCTGAAGACGACGATGACAATGATGCGGCCATC
AGTATGATCAGAAAAATGTTTGGATACAATCCGCAAAAGTTTGCTGGTCG
TGATGAAGACGACAGTGATATGGAAGCAAACTTTGACGACATCATGAGGG
AAGAAAAGAGGAGTGCGAGAATCGCCCAAAAGGAGGACGAGGAACAGCTG
CGCTTGATAGAGGAGGAGGAAAGGCGCGAACGCGAACGGATAAGAAAAAA
GCGGAAGATGGGATAA
SEQ ID NO: 3:
an amino acid sequence of the encoded protein of
the pomegranate PgIAA9A gene
MSPPLLGAEEGGTSDVTKVAGSPSSKDSPDGLGLGLKERNYLGLSDCSSV
DSSTVSGQSDPEAQNNKLNLKATELRLGLPGSQSPERDLDEKPLFPLLPS
KDGIKTVVSGNKRGFSDAIDGFSDKKWMFHSAGTDEGTQPLGQGKFPANA
GPKQPATKEVPAKPLPERANGINPSRAANNAPAAKAQVIGWPPVRSFRKN
TLATTSKDNDEVDGKPGPGALFIKVSMDGAPYLRKVDLRNYSKYYELSVA
LEKMFSGFTIGQCGTNGTGGRESMSESKLKDLLHGSDYVLTYEDKDGDWM
LVGDVPWECPLPPLLAASALSGIEISDRRASASSIQLAAISQFSLVRFQE
IGSRMRGYEDDEYEDYDNYEEEGYEQEEGGDEEDEYEEEEEEPKPTKEEL
EYLELRQKLKESIRKRMKKAASGSEEIKKDNYGSFFGPSKPVIAQRVIQE
SKSLLENQHLAARLLKSHQNGNKSSSSRPDGSKPGVRPQAPRVSQLKQRV
QKIKDTRDYSFLLSDDAELPASPKQPPPRSIPAPSLDARSAKLSSQSRLN
PGSNSRHVNGGREERRPASSNRHSNPRPGSSNGHSNNRPGSANGYSNTRP
GSANGHSNLRPGSANVHSNLRPGSANGHSNLRPGSANGHSNLRPGSANGH
SNLGAGPNKLSSASKPQMAANRKQLGSNSGNGPGRPAVSNGLQPKKPIPP
PQRSSQGLKSSVPAGVKPPATKMQSSASKYQVEPRRNVQQPNKGKILSKH
SVASTRPQVQAQMSKPQKQIPSHNRLQDQRPKKRPSRPFPEDDDDNDAAI
SMIRKMFGYNPQKFAGRDEDDSDMEANFDDIMREEKRSARIAQKEDEEQL
RLIEEEERRERERIRKKRKMG
SEQ ID NO: 4:
an amino acid sequence of the encoded protein of
the pomegranate PgIAA9Am gene
MSPPLLGAEEGGTSDVTKVAGSPSSKDSPDGLGLGLKERNYLGLSDCSSV
DSSTVSGQSDPEAQNNKLNLKATELRLGLPGSQSPERDLDEKPLFPLLPS
KDGIKTVVSGNKRGFSDAIDGFSDKKWMFHSAGTDEGTQPLGQGKFPANA
GPKQPATKEVPAKPLPERANGINPSRAANNAPAAKAQVIGWSPVRSFRKN
TLATTSKDNDEVDGKPGPGALFIKVSMDGAPYLRKVDLRNYSKYYELSVA
LEKMFSGFTIGQCGTNGTGGRESMSESKLKDLLHGSDYVLTYEDKDGDWM
LVGDVPWECPLPPLLAASALSGIEISDRRASASSIQLAAISQFSLVRFQE
IGSRMRGYEDDEYEDYDNYEEEGYEQEEGGDEEDEYEEEEEEPKPTKEEL
EYLELRQKLKESIRKRMKKAASGSEEIKKDNYGSFFGPSKPVIAQRVIQE
SKSLLENQHLAARLLKSHQNGNKSSSSRPDGSKPGVRPQAPRVSQLKQRV
QKIKDTRDYSFLLSDDAELPASPKQPPPRSIPAPSLDARSAKLSSQSRLN
PGSNSRHVNGGREERRPASSNRHSNPRPGSSNGHSNNRPGSANGYSNTRP
GSANGHSNLRPGSANVHSNLRPGSANGHSNLRPGSANGHSNLRPGSANGH
SNLGAGPNKLSSASKPQMAANRKQLGSNSGNGPGRPAVSNGLQPKKPIPP
PQRSSQGLKSSVPAGVKPPATKMQSSASKYQVEPRRNVQQPNKGKILSKH
SVASTRPQVQAQMSKPQKQIPSHNRLQDQRPKKRPSRPFPEDDDDNDAAI
SMIRKMFGYNPQKFAGRDEDDSDMEANFDDIMREEKRSARIAQKEDEEQL
RLIEEEERRERERIRKKRKMG
SEQ ID NO: 5:
PgIAA9A-F
ATGTCTCCACCGCTCCTGGGT
SEQ ID NO: 6:
PgIAA9A-R
TCAGTTTCTTGCCTTGGATTTCT
SEQ ID NO: 7:
PgIAA9Am-F
CAGGTCATTGGTTGGTCTCCAGTGAGATCCTTCAG
SEQ ID NO: 8:
PgIAA9Am-R
CTGAAGGATCTCACTGGAGACCAACCAATGACCTG
SEQ ID NO: 9:
PgIAA9A-1300GFP-F
CACGGGGGACGAGCTCGGTACCATGTCTCCACCGCTCCTGGGT
SEQ ID NO: 10:
PgIAA9A-1300GFP-R
CCATGTCGACTCTAGAGGATCCTCAGTTTCTTGCCTTGGATTTCT
SEQ ID NO: 11:
PgActin7-F
TATTGTTGGTCGTCCCAGGC
SEQ ID NO: 12:
PgActin7-R
CCTCTCTTCGATTGGGCCTC
SEQ IDNO: 13:
AtActin2-F
GGTAACATTGTGCTCAGTGGTGG
SEQ ID NO: 14:
AtActin2-R
AACGACCTTAATCTTCATGCTGC
SEQ ID NO: 15:
PgIAA9AqRT-PCR-F
AACTACCTTGGACTTTCCGACTG
SEQ ID NO: 16:
PgIAA9AqRT-PCR-R
TGTGGCCTTCAGGTTCAGC

What is claimed is:

1. A pomegranate PgIAA9A gene, wherein a nucleotide sequence of the pomegranate PgIAA9A gene is shown in SEQ ID NO:1.

2. An encoded protein of a pomegranate PgIAA9A gene, wherein an amino acid sequence of the encoded protein is shown in SEQ ID NO:3.

3. An application of the pomegranate PgIAA9A gene of claim 1 for regulating plant seed growth and development.

4. An application of the pomegranate PgIAA9A gene of claim 1 for negatively regulating plant seed size and lignin accumulation.

5. An application of the pomegranate PgIAA9A gene of claim 1 for negatively regulating seed coat size and lignin accumulation of pomegranate.

6. The application of claim 3, wherein during the application, the pomegranate PgIAA9A gene is point-mutated into a PgIAA9Am gene, wherein a nucleotide sequence of the PgIAA9Am gene is shown in SEQ ID NO:2.

7. The application of claim 6, wherein an amino acid sequence of an encoded protein of the PgIAA9Am gene is shown in SEQ ID NO:4.

8. The application of claim 4, wherein during the application, the pomegranate PgIAA9A gene is point-mutated into a PgIAA9Am gene, wherein a nucleotide sequence of the PgIAA9Am gene is shown in SEQ ID NO:2.

9. The application of claim 8, wherein an amino acid sequence of an encoded protein of the PgIAA9Am gene is shown in SEQ ID NO:4.

10. The application of claim 5, wherein during the application, the pomegranate PgIAA9A gene is point-mutated into a PgIAA9Am gene, wherein a nucleotide sequence of the PgIAA9Am gene is shown in SEQ ID NO:2.

11. The application of claim 10, wherein an amino acid sequence of an encoded protein of the PgIAA9Am gene is shown in SEQ ID NO:4.
